(19) United States
(12) Patent Application Publication
(10) Pub. No.: US 2022/0313766 A1
(43) Pub. Date: Oct. 6, 2022

(54) Composition Comprising Cannabis Extracts and Method of Manufacturing Same

(71) Applicant: MEC11S Inc., Chatham (CA)

(72) Inventor: Eric GAUDRAULT, Chatham (CA)

(21) Appl. No.: 17/713,608

(22) Filed: Apr. 5, 2022

(30) Foreign Application Priority Data
Apr. 5, 2021 (CA) 3114085

(51) Int. Cl. A61K 36/185; A23L 33/105; A23L 29/212; A61K 9/20

(52) U.S. Cl. CPC A61K 36/185; A23L 33/105; A23L 29/212; A61K 9/2095; A23V 2002/00; A61K 2236/53; A61K 2236/51

(57) ABSTRACT

A composition incorporating cannabis extracts and methods of manufacturing same utilize a powder that is formed from vapour or oil that has been extracted from a cannabis plant. The oil may be mixed with a thickener to form the powder. Alternatively, the vapour may be extracted from the cannabis plant and collected in a filter medium, which is then dehydrated and ground into a powder. The vapour may be extracted in a chamber at high temperature, and may then be condensed after extraction in a chamber at low temperature, which may also be a vacuum chamber. The dehydration of the filter medium may take place under a UV light in a chamber kept at about 60° C. The potency of the product may be tested at various intermediate steps in the manufacturing process. The composition is preferably in the form of a pill.

### CROSS-REFERENCES TO RELATED APPLICATIONS

This application claims the benefit of Canadian Patent Application No. 3,114,085, filed Apr. 5, 2021, the entire content of which is herein incorporated by reference.

### TECHNICAL FIELD

The present disclosure relates generally to compositions and methods of manufacturing same, and more particularly relates to a pill comprising cannabis extracts and methods of manufacturing same.

### BACKGROUND

Previously, there were a handful of ways to consume cannabis for either recreational or medical purposes, including inhalation or smoking in the form of cigarettes, water pipes, and vaporizers, as well as consumption in the form of an oil or as part of another edible product.

All of these previously known methods have drawbacks. For example, smoking or inhaling cannabis can lead to an irritation in the user's throat, and preparing a device for smoking or inhaling can be messy and time consuming. These methods can also lead to unpleasant odours and are not as discrete as other methods of consuming cannabis.

While consuming cannabis in the form of oils or edibles mitigates some of the above issues associated with smoking or inhaling, they are expensive, and it is difficult to determine the appropriate dosage. There can also be unpleasant tastes associated with consuming cannabis as an oil or an edible.

The above methods of consuming cannabis also force the user to select the appropriate dose of cannabis to consume at a given time, which can be difficult to do. As a result, it is difficult to ascertain a standard dose when obtaining cannabis products.

Therefore, there is a need for a product comprising cannabis that alleviates the issues associated with the above-mentioned methods of consuming cannabis.

### SUMMARY OF THE DISCLOSURE

The present disclosure relates to a composition comprising cannabis extracts and methods of manufacturing same. The composition comprises a powder that is formed from vapour or oil that has been extracted from a cannabis plant. The oil may be mixed with a thickener to form the powder. Alternatively, the vapour may be extracted from the cannabis plant and collected in a filter medium, which is then dehydrated and ground into a powder. The vapour may be extracted in a chamber at high temperature, and may then be condensed after extraction in a chamber at low temperature, which may also be a vacuum chamber. The dehydration of the filter medium may take place under a UV light in a chamber kept at about 60° C. The potency of the product may be tested at various intermediate steps in the manufacturing process. The composition is preferably in the form of a pill.

According to one aspect, there is provided a method of manufacturing a composition comprising extracts from a cannabis plant, the method comprising the steps of extracting a vapour from the cannabis plant, collecting the vapour in a filter medium, dehydrating the filter medium, and grinding the filter medium into a powder.

According to another aspect, the method further comprises the step of compressing the powder into a pill.

According to another aspect, the method further comprises the step of automatically releasing the vapour from a first chamber after extraction from the cannabis plant, wherein the vapour is automatically released upon reaching a pre-determined temperature and humidity level in the first chamber.

According to another aspect, the method further comprises the step of condensing the vapour after extraction.

According to another aspect, the vapour is condensed in a second chamber comprising the filter medium.

According to another aspect, the vapour is condensed at a temperature of about −40° C.

According to another aspect, the vapour is condensed in a vacuum chamber.

According to another aspect, the vapour is extracted from the cannabis plant at a temperature of about 225° C.

According to another aspect, the method further comprises the step of testing the potency of the cannabis plant prior to the extraction of the vapour.

According to another aspect, the filter medium is dehydrated at a temperature of about 60° C.

According to another aspect, the dehydration of the filter medium is done under a UV light.

According to another aspect, the method further comprises the step of testing the potency of the filter medium prior to dehydration.

According to another aspect, the method further comprises the step of testing the potency of the powder prior to compressing the powder into the pill.

According to another aspect, the method further comprises the step of applying a coating to the pill.

According to an alternative aspect, there is provided a method of manufacturing a composition comprising extracts from a cannabis plant, the method comprising the steps of providing an oil extracted from the cannabis plant, and mixing the oil with a thickener to form a powder.

According to another aspect, the method further comprises the step of compressing the powder into a pill.

According to another aspect, the method further comprises the step of testing the potency of the oil.

According to another aspect, the thickener is maltodextrin.

According to another aspect, the method further comprises the step of filtering the powder prior to compressing the powder into the pill.

According to another aspect, the method further comprises the step of testing the potency of the powder prior to compressing the powder into the pill.

According to another aspect, the method further comprises the step of applying a coating to the pill.

According to another aspect, there is provided a pill comprising extracts from a cannabis plant made by any of the above methods.

### DESCRIPTION OF THE DRAWINGS

FIG. 1 is a process diagram for manufacturing a pill comprising cannabis in accordance with an embodiment of the present disclosure.

FIG. 2 is a process diagram for manufacturing a pill comprising cannabis in accordance with another embodiment of the present disclosure.

### DETAILED DESCRIPTION

The present disclosure relates to a composition comprising cannabis extracts and methods of manufacturing same. The composition comprises a powder that is formed from vapour or oil that has been extracted from a cannabis plant. The oil may be mixed with a thickener to form the powder. Alternatively, the vapour may be extracted from the cannabis plant and collected in a filter medium, which is then dehydrated and ground into a powder. The vapour may be extracted in a chamber at high temperature, and may then be condensed after extraction in a chamber at low temperature, which may also be a vacuum chamber. The dehydration of the filter medium may take place under a UV light in a chamber kept at about 60° C. The potency of the product may be tested at various intermediate steps in the manufacturing process. The composition is preferably in the form of a pill.

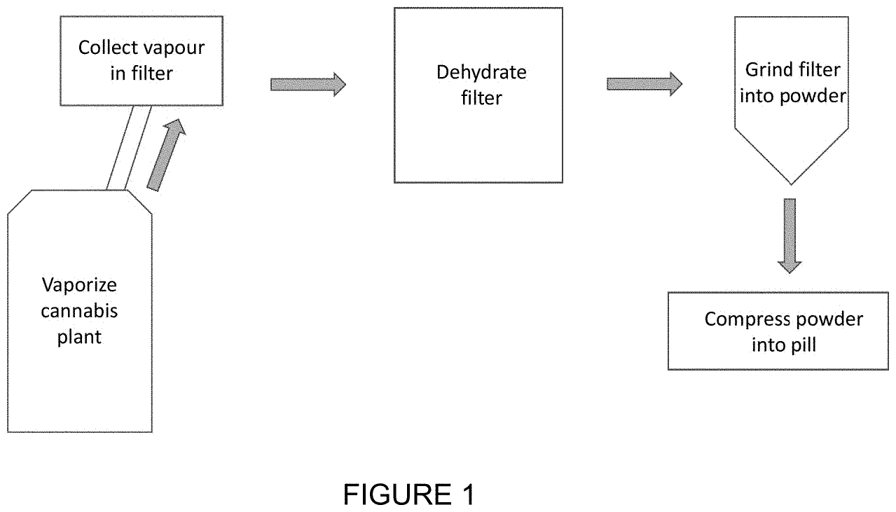

Referring to FIG. 1, a method for manufacturing a pill comprising extracts from a cannabis plant is shown. First, a vapour is extracted from the cannabis plant. Next, the vapour is collected in a filter medium. The filter medium is then dehydrated, following which the dehydrated filter medium is ground into a powder. Finally, the powder is compressed into a pill.

Considering each of these steps in turn, a cannabis plant may be placed into a pressure cooker, vaporizer, or other pressurized and/or heated chamber in order to extract a vapour from the cannabis plant. In a preferred embodiment, the cannabis plant will be freshly harvested. Preferably, the vapour extraction will be done at a high temperature, most preferably at about 225° C. Based on the boiling points of the various cannabinoids and terpenes contained in a typical cannabis plant, this will maximize the cannabis extracts included in the vapour.

Further, in an embodiment of the present invention, the potency of the cannabis plant may be tested prior to being vaporized. A person skilled in the art will appreciate that there are known methods of testing the potency of a cannabis plant.

The next step is to collect the vapour in a filter medium. The filter medium can be a common filter such as rice paper, or any other appropriate filter known to a person skilled in the art. The vapour may be collected in the filter medium by any known method, but it will preferably be done by exhausting the vapour from the pressurized and/or heated chamber into a separate chamber comprising the filter medium.

In a preferred embodiment, a pressure release valve may be present on the pressurized and/or heated chamber to automatically release the vapour once a pre-determined temperature and humidity level are reached. This will aid in preventing the vapour from condensing prior to being exhausted from the pressurized and/or heated chamber. For the same reason, the pressurized and/or heated chamber may be lined with porcelain, or another material known to a person skilled in the art, to reduce the likelihood of vapour condensing to the pressurized and/or heated chamber.

The chamber comprising the filter medium may be a condenser. In this embodiment, the condenser may operate at a low temperature, preferably at about −40° C. The chamber comprising the filter medium may also be a vacuum chamber. Both a low temperature and a vacuum will aid in the rapid condensation and binding of the vapour to the filter medium.

Once the vapour is collected in the filter medium, the filter medium may go through a dehydration process. The dehydration may occur in a chamber that is maintained at a temperature of about 60° C. In a preferred embodiment, the dehydration process may occur under a UV light to aid in the breakdown of the oils in the vapour. At the end of the dehydration process, the filter medium comprising the cannabis extracts is preferably dry and brittle.

In an embodiment of the present invention, the potency of the vapour collected in the filter medium may be tested prior to the filter medium being dehydrated. A person skilled in the art will appreciate that there are known methods of testing the potency of vapour extracted from a cannabis plant.

After the filter medium has undergone the dehydration process, it may be ground into a flour-like compound, or powder. Preferably, the grinding is done slowly so as not to introduce heat and moisture into the process.

In an embodiment of the present invention, the potency of the powder may be tested prior to compressing the powder into pills. This will aid in calibrating the compressor for consistent and accurate dosages in the pills. A person skilled in the art will appreciate that there are known methods of testing the potency of powder comprising extracts from a cannabis plant.

The powder may then be compressed into pills using a standard machine known to persons skilled in the art. Preferably, a coating may be applied to the pills, including a candy coating, an enteric coating, or any other type of coating known to a person skilled in the art.

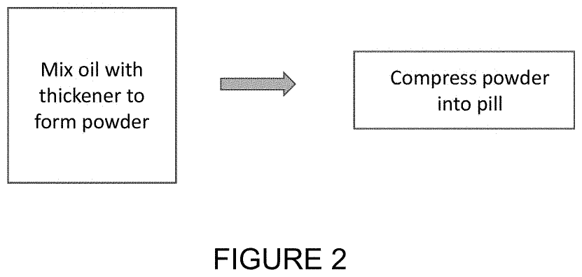

Referring to FIG. 2, an alternative method for manufacturing a pill comprising extracts from a cannabis plant is shown. First, there is provided an oil that has been extracted from a cannabis plant. The oil is mixed with a thickener to form a powder, and the powder is compressed into a pill.

In an embodiment of the present invention, the potency of the oil may be tested prior to being mixed with a thickener. A person skilled in the art will appreciate that there are known methods of testing the potency of oil extracted from a cannabis plant.

The thickener may be of any type known to a person skilled in the art, but is preferably maltodextrin. Once the oil and the thickener are mixed together to form a powder, the powder may be filtered through a sieve or other appropriate filter device to remove any agglomerations prior to being compressed into pills.

In an embodiment of the present invention, the potency of the powder may be tested prior to compressing the powder into pills. This will aid in calibrating the compressor for consistent and accurate dosages in the pills. A person skilled in the art will appreciate that there are known methods of testing the potency of powder comprising extracts from a cannabis plant.

The powder may be compressed into pills using a standard machine known to persons skilled in the art. Preferably, a coating may be applied to the pills, including a candy coating, an enteric coating, or any other type of coating known to a person skilled in the art.

The composition comprising cannabis has been described in the above illustrative embodiments as being preferably provided in the form of a pill. However, the described embodiments contemplate alternative dosage forms and routes of administration for the composition, all of which would be known to the person skilled in the art, and which would include tablets, capsules, and gels. Any of these dosage forms and routes of administration may further comprise flavourings, binders, excipients, fillers, and other ingredients known to a person skilled in the art.

Although the invention has been described with reference to illustrative embodiments, it is to be understood that the invention is not limited to these precise embodiments. Numerous modifications, variations, and adaptations may be made to the particular embodiments of the invention described above without departing from the scope of the invention. The scope of the claims should not be limited by the preferred embodiments set forth in the examples, but should be given the broadest interpretation consistent with the description as a whole.

Immaterial modifications may be made to the embodiments described herein without departing from what is covered by the claims. The indefinite article “a” before a claim feature does not exclude more than one of the feature being present. Each of the individual features described here may be used in one or more embodiments and is not, by virtue only of being described here, to be construed as essential to all embodiments as defined by the claims.

1. A method of manufacturing a composition comprising extracts from a cannabis plant, the method comprising the steps of:
extracting a vapour from the cannabis plant;
collecting the vapour in a filter medium;
dehydrating the filter medium; and
grinding the filter medium into a powder.

2. The method of claim 1, further comprising the step of compressing the powder into a pill.

3. The method of claim 1, further comprising the step of automatically releasing the vapour from a first chamber after extraction from the cannabis plant, wherein the vapour is automatically released upon reaching a pre-determined temperature and humidity level in the first chamber.

4. The method of claim 1, further comprising the step of condensing the vapour after extraction.

5. The method of claim 4, wherein the vapour is condensed in a second chamber comprising the filter medium.

6. The method of claim 4, wherein the vapour is condensed at a temperature of about −40° C.

7. The method of claim 4, wherein the vapour is condensed in a vacuum chamber.

8. The method of claim 1, wherein the vapour is extracted from the cannabis plant at a temperature of about 225° C.

9. The method of claim 1, further comprising the step of testing the potency of the cannabis plant prior to the extraction of the vapour.

10. The method of claim 1, wherein the filter medium is dehydrated at a temperature of about 60° C.

11. The method of claim 1, wherein the dehydration of the filter medium is done under a UV light.

12. The method of claim 1, further comprising the step of testing the potency of the filter medium prior to dehydration.

13. The method of claim 2, further comprising the step of testing the potency of the powder prior to compressing the powder into the pill.

14. The method of claim 2, further comprising the step of applying a coating to the pill.

15. A method of manufacturing a composition comprising extracts from a cannabis plant, the method comprising the steps of:
providing an oil extracted from the cannabis plant; and
mixing the oil with a thickener to form a powder.

16. The method of claim 15, further comprising the step of compressing the powder into a pill.

17. The method of claim 15, further comprising the step of testing the potency of the oil.

18. The method of claim 15, wherein the thickener is maltodextrin.

19. The method of claim 16, further comprising the step of filtering the powder prior to compressing the powder into the pill.

20. The method of claim 16, further comprising the step of testing the potency of the powder prior to compressing the powder into the pill.

21. The method of claim 16, further comprising the step of applying a coating to the pill.

22. A pill comprising extracts from a cannabis plant made by the method of claim 2.

23. A pill comprising extracts from a cannabis plant made by the method of claim 16.
